(19) United States
(12) Patent Application Publication
(10) Pub. No.: US 2011/0212137 A1
(43) Pub. Date: Sep. 1, 2011

(54) INHALABLE PHARMACEUTICAL COMPOSITION

(75) Inventors: Paul Michael Dorinsky, Blue Bell, PA (US); David Price, Aylsham (GB)

(73) Assignee: TEVA BRANDED PHARMACEUTICAL PRODUCTS R&D, INC., Horsham, PA (US)

(21) Appl. No.: 13/027,371

(22) Filed: Feb. 15, 2011

Related U.S. Application Data
(60) Provisional application No. 61/304,998, filed on Feb. 16, 2010.

(51) Int. Cl. A61K 9/12; A61K 31/573; A61P 11/06

(52) U.S. Cl. 424/400; 514/180

(57) ABSTRACT

An inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm, for maintaining lung function above 60% of baseline FEV₁, or reducing the occurrences of lung function falling below 60% of baseline FEV₁.

### CROSS REFERENCE TO RELATED APPLICATION

This application is related to and claims the benefit of U.S. Provisional Application No. 61/304,998, filed on Feb. 16, 2010, the contents of which are incorporated herein by reference.

### TECHNICAL FIELD OF THE INVENTION

This invention relates to an inhalable pharmaceutical composition, and particularly to an inhalable pharmaceutical solution aerosol comprising beclometasone dipropionate.

### BACKGROUND OF THE INVENTION

Asthma is a common inflammatory disease in which the airways of the respiratory system become chronically narrowed and constricted by oedema (fluid retention). During an attack, a patient suffers from laboured breathing accompanied especially by wheezing and coughing and by a sense of constriction in the chest due to bronchospasm (sudden constriction of the muscles in the walls of the bronchioles), mucosal oedema and mucus formation. Asthma is triggered by hyperreactivity to various stimuli (such as allergens or a rapid change in air temperature). In sensitised individuals, inhaled allergens (allergy triggers) provoke a hyperimmune response characterised by recruitment of immune cells and production of immunoglobulin E (IgE) antibodies. Asthma is caused by the attachment of IgE antibodies to mast cells (a resident cell of several types of tissues throughout the body, particularly in proximity to surfaces that interface the external environment). This attachment activates mast cells and on renewed exposure to the same antigen, degranulation of the mast cells occurs, leading to the rapid release of inflammatory mediators, such as histamine, proteoglycans, and cytokines. Asthma is a result of localised release of such mediators.

In a patient suffering from asthma, airflow obstruction is largely reversible and the patient experiences a significant response to inhaled bronchodilators and inhaled antiinflammatories.

Asthma is generally treated using a combination of long-term treatment and short-term episodic treatment of acute attacks. Long-term treatment of asthma involves the use of antiinflammatories and long-acting bronchodilators. Short-term episodic treatment employs short-acting bronchodilators. Inhaled glucocorticosteroids (ICS) are a feature of the currently preferred treatment for moderate to severe allergic asthma, and have been shown to act by suppressing the adaptive immune response while not suppressing innate immune response. There are various effective ICSs available in the art, for example, fluticasone propionate or CFC-beclometasone (a formulation comprising beclometasone dipropionate and a chlorofluorocarbon propellant).

Beclometasone dipropionate (INN), also known as beclomethasone dipropionate (USAN) or (8S,9R,10S,11S,13S,14S,16S,17R)-9-chloro-11-hydroxy-10,13,16-trimethyl-3-oxo-17-[2-(propionyloxy)acetyl]-6,7,8,9,10,11,12,13,14,15,16,17-dodecahydro-3H-cyclopenta[a]phenanthren-17-yl propionate (IUPAC), is the subject of the present invention.

Formulations of beclometasone dipropionate are known in the art. For example, U.S. Pat. No. 5,776,432 discloses a pharmaceutical solution aerosol formulation comprising beclometasone dipropionate, a hydrofluorocarbon propellant selected from 1,1,1,2-tetrafluoroethane (norflurane or propellant 134a), 1,1,1,2,3,3,3-heptafluoropropane (propellant 227) and a mixture thereof, and ethanol (anhydrous) to solubilise the beclometasone dipropionate in the propellant. The beclometasone dipropionate is dissolved in the formulation, and the formulation is substantially free of surfactant.

However, further improvements in treatment are desired for the effective management of asthma.

### SUMMARY OF THE INVENTION

Accordingly, the present invention provides an inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm, for maintaining lung function above 60% of baseline FEV₁, or reducing the occurrences of lung function falling below 60% of baseline FEV₁.

This formulation provides a surprising improvement in the lung (pulmonary) function compared to other known ICSs.

### BRIEF DESCRIPTION OF THE DRAWINGS

FIG. 1 shows in schematic form the study design used in the trials described in the Example.

FIG. 2 shows the adjusted odds ratio for successful asthma control observed for various corticosteroids in the trials described in the Example.

FIG. 3 shows the adjusted odds ratio (revised definition) for successful asthma control observed for various corticosteroids in the trials described in the Example.

FIG. 4 shows the adjusted rate ratio for exacerbation rate observed for various corticosteroids in the trials described in the Example.

### DETAILED DESCRIPTION OF THE INVENTION

The inhalable pharmaceutical solution aerosol of the present invention comprises beclometasone dipropionate, ethanol and a propellant selected from 1,1,1,2-tetrafluoroethane, 1,1,1,2,3,3,3-heptafluoropropane and a mixture thereof.

The active ingredient beclometasone dipropionate is generally present in a formulation of the invention in a therapeutically effective amount, i.e. an amount such that metered volumes of the medicament administered to the patient contains an amount of drug effective to exert the intended therapeutic action. The aerosol solution preferably contains 0.02 to 0.6 percent by weight, more preferably 0.05 to 0.5 percent by weight of beclometasone dipropionate, based on the total weight of the solution.

Ethanol is present in an amount effective to solubilise the beclometasone dipropionate in the propellant. Preferably, the solution contains 1 to 20 percent by weight of ethanol, more preferably 2 to 12 percent by weight and most preferably 4 to 10 percent by weight, based on the total weight of the aerosol solution. The ethanol will be present in an amount sufficient to dissolve substantially all of the medicament present in the formulation and to maintain the medicament dissolved over the time period and conditions experienced by commercial aerosol products. Preferably the ethanol is present in an amount to prevent precipitation of the active ingredient even at temperatures down to −20° C. The ethanol is preferably anhydrous ethanol, although trace amounts of water absorbed by the ingredients, for example during manufacture of the medicament, may be tolerated.

The hydrofluorocarbon propellant may be propellant 134a, propellant 227 or a mixture thereof. The solution preferably contains 80 to 99 percent by weight of propellant, more preferably 88 to 98 percent by weight, and most preferably 90 to 95 percent by weight, based on the total weight of the aerosol solution. The hydrofluorocarbon propellant is preferably the only propellant present in the formulations of the invention.

The solution of the present invention is preferably substantially free of surfactant. Surfactants are often added to suspensions to stabilise the suspension. However, since the formulation of the present invention is a solution, a surfactant is not required. Nevertheless, small quantities can be tolerated without adversely affecting the formulation. Preferably the formulation contains no more than 0.0005 percent by weight of a surfactant based on the total weight of the solution. Preferred formulations contain no surfactant. Presence of a significant amount of a surfactant is believed to be undesirable for solution formulations of beclometasone dipropionate because surfactants such as oleic acid and lecithin are believed to promote chemical degradation of the active ingredient when the latter is dissolved in the mixture of the propellant and ethanol.

A preferred solution according to the present invention comprises 0.02 to 0.6 percent by weight beclometasone dipropionate, 1 to 20 percent by weight ethanol and 80 to 99 percent by weight of propellant, wherein the percentages by weight are based on the total weight of the solution aerosol. A particularly preferred solution consists essentially of beclometasone dipropionate, ethanol and a propellant selected from 1,1,1,2-tetrafluoroethane, 1,1,1,2,3,3,3-heptafluoropropane and a mixture thereof; more preferably the solution consists of these components.

The solution of the present invention may be prepared by dissolving the desired amount of beclometasone dipropionate in the desired amount of ethanol accompanied by stirring or sonication. An aerosol container may then be filled using conventional cold-fill or pressure-fill methods.

The solution aerosol of the present invention is administered from a pressurised metered-dose inhaler (pMDI). A pMDI has two key components, namely a canister and an actuator (or mouthpiece). The canister has a vial for storing the solution coupled to a metering dose valve having an actuating stem. The container is housed in an actuator where the actuating step is in fluid communication with a discharge nozzle in the actuator. Actuation of the device releases a single metered dose of solution aerosol. The aerosol passes through the discharge nozzle resulting in a breaking up of the volatile propellant into droplets, followed by rapid evaporation of these droplets as they are inhaled into the lungs. The discharge nozzle preferably has an orifice diameter of 100-300 μm, more preferably 150-250 μm and most preferably 248 μm. This orifice size encourages the formation of droplets of the required size in the aerosol solution of the present invention.

The solution of the present invention when administered from the pMDI forms a fine aerosol of droplets which has a droplet size having a mass median aerodynamic diameter of 0.5-2.0 μm and preferably 0.8-1.2 μm. This small droplet size facilitates deep lung penetration. The droplet size may be measured using conventional techniques, such as using a Next Generation Pharmaceutical Impactor (NGI) or an Andersen eight-stage impactor (ACI).

It has been found that the solution aerosol of the present invention is capable of providing improved lung function and is able to maintain lung function above 60% of baseline FEV₁, or to reduce the occurrences of lung function falling below 60% of baseline FEV₁ in a patient suffering from asthma. Preferably the solution aerosol of the present invention may be used for maintaining lung function above 70% of baseline FEV₁, or reducing the occurrences of lung function falling below 70% of baseline FEV₁, and more preferably for maintaining lung function above 80% of baseline FEV₁, or reducing the occurrences of lung function falling below 80% of baseline FEV₁. Reducing the occurrences is compared to a patient taking no ICS or even other ICS, such as fluticasone propionate and/or CFC-beclometasone dipropionate.

FEV₁ is the maximal volume of air exhaled in the first second of a forced expiration from a position of full inspiration, expressed in litres at body temperature and ambient pressure saturated with water vapour (BTPS). FEV₁ is determined by spirometry which is the most common of the Pulmonary Function Tests (PFTs). FEV₁ is measured according to the parameters set down in the joint statements on lung function testing by the American Thoracic Society (ATS) and the European Respiratory Society (ERS), see the Series “ATS/ERS Task Force: Standardisation of Lung Function Testing, Edited by V. Brusasco, R. Crapo and G. Viegi, Numbers 1-5, Eur. Respir. J. 2005; 26: 153-161,319-338,511-522,720-735 and 948-968.

The FEV₁ obtained is expressed in litres for an individual patient. It must then be compared to a reference (predicted) value based on healthy subjects. Predicted values are obtained from studies of normal, healthy subjects with the same anthropometric (sex, age and height) and ethnic characteristics of the patient being tested. Reference values are derived from large groups of volunteers. The criteria to define subjects as normal or healthy have been laid down the ATS and ERS.

The result is an FEV₁ expressed as a percentage of predicted. The severity of the asthma is defined by this percentage. The ATS/ERS statements lay down the following definitions: mild asthma is an FEV₁ of 70% of predicted or above; moderate 60-69% of predicted; moderately severe 50-59% of predicted; severe 35-49% of predicted and very severe less than 35% of predicted The percentage provides a baseline measurement for any given patient. It has been found that the solution aerosol of the present invention maintains the baseline for a given patient within the above-described limits.

A group of patients has also been identified which receive particular benefit from the solution aerosol of the present invention. That is, a patient showing insufficient response to inhalable fluticasone propionate and/or an inhalable formulation comprising beclometasone dipropionate and a chlorofluorocarbon propellant. An insufficient response herein is defined as failing to maintain the baseline for a given patient within the above-described limits.

The present invention also provides a method for maintaining lung function above 60% of baseline FEV₁, or reducing the occurrences of lung function falling below 60% of baseline FEV₁ by administering to a patient in need thereof, an inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm.

The present invention will now be described with reference to the following example, which is not intended to be limiting.

### EXAMPLE

Trials were conducted using Qvar® which is a solution aerosol formulation containing beclometasone dipropionate, anhydrous ethanol, and propellant 134a (1,1,1,2-tetrafluoroethane). Qvar® is available in two doses, 50 μg and 100 μs. The two formulations contain: beclometasone dipropionate 0.05 or 0.10 mg per actuation, anhydrous ethanol 4.74 mg per actuation and propellant 134a 54.51 or 54.46 mg per actuation. Both formulations have a density at 20° C. of 1.166 g/mL and a MMAD of 1.1 μm.

Qvar® and other inhaled corticosteroids (ICS) CFC-beclometasone (BDP) and fluticasone propionate (FP) were compared in a large representative, UK clinical database.

Asthma patients, aged 5-60, without other chronic respiratory diseases, receiving Qvar®, CFC-BDP or FP MDIs (Multiple Dose Inhalers) who subsequently had an ICS increase between January 1997-June 2006 were identified from UK General Practice Research Database. Patients were eligible if they had data available for the twelve months prior to AND twelve months after the date of ICS increase. Multiple logistic regression, adjusted for baseline severity, provided odds of successful asthma control (no asthma hospital attendances, oral steroids, consultations or hospital admissions for lower respiratory tract infections requiring antibiotics) during 12 month follow-up. Poisson regression was used to compare asthma exacerbation rates (unscheduled hospital admissions/A&E attendances for asthma or use of oral steroids).

Data were available for 4133 patients (8% Qvar®, 15% FP, 77% BDP). Odds ratio (95% CI) for success was lower with BDP and FP compared with Qvar® (0.65 [0.47-0.90] and 0.71 [0.50-1.02] respectively). Exacerbation rates (95% CI) were higher with BDP and FP 1.44 (0.94-2.21) and 1.61 (1.02-2.55), respectively.

Patients receiving increased ICS therapy with Qvar® were more likely to achieve successful asthma control and less likely to experience exacerbations.

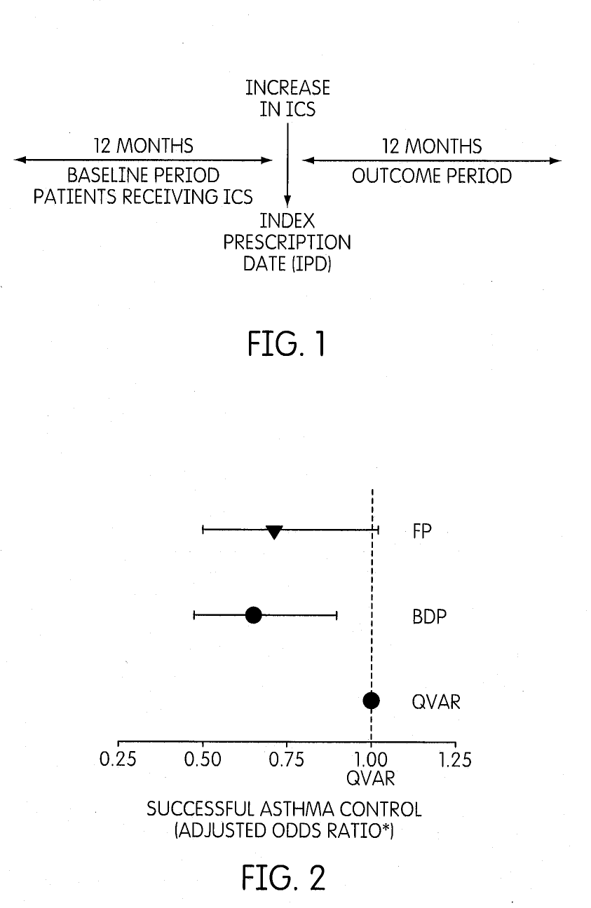

Study Design: A retrospective, observational, cohort study design was used, consisting of two time periods: a baseline period for confounder definition (minimum of one year before index prescription date) and an outcome period of one year post-index prescription date. The study design is shown in schematic format in FIG. 1.

Statistical Analysis: Descriptive statistics were used to compare baseline outcomes. Multiple logistic regression, adjusted for baseline severity and other potential confounders found at baseline, was used to determine the odds of successful asthma control. Poisson regression was used to compare exacerbation rates.

### Outcomes

Primary Outcome: Proxy for successful asthma control defined as no asthma hospital attendances (including outpatients, A&E or inpatient attendances), acute courses of oral steroids, GP consultations or hospital admissions for lower respiratory tract infections requiring antibiotics during 12-month follow-up.

Secondary Primary Outcome: Proxy for successful asthma control defined as above with the additional criteria of average daily prescribed dose of salbutamol of no more than 200 mcg and terbutaline 500 mcg (SABA).

Secondary Outcome: Asthma exacerbation rates (defined as unscheduled hospital admissions, A&E attendances for asthma, or use of oral steroids).

### Results

As used herein, the term “adjusted” means adjusted for Beta Blockers (Y/N), GERD Diagnosis (Y/N), Cardiac Disease Diagnosis (Y/N), CCI Score, NSAIDs (Y/N), paracetamol (Y/N), Age (5-12, 13-20, 21-30, 31-40, 41-50, 51-60), Gender, Asthma Scripts, Asthma Consultations, Total Oral Steroids (0, 1, 2, 3+), SABA Dosage (0 mcg, >0-100 mcg, >100-200 mcg, >200-400 mcg, >400-800 mcg, >800 mcg), Antibiotics, Planned OPD, Asthma Baseline Status (successful/unsuccessful), Year of Index Prescription Date, Average ICS Daily Dose in Baseline Period.

Predefined Proxy of Successful Asthma Control: In terms of adjusted successful asthma control, Qvar® was significantly more effective than BDP (OR: 0.65; 95% CI: 0.47-0.90), and numerically more effective than FP (OR: 0.71; 95% CI: 0.50-1.02). See FIG. 2.

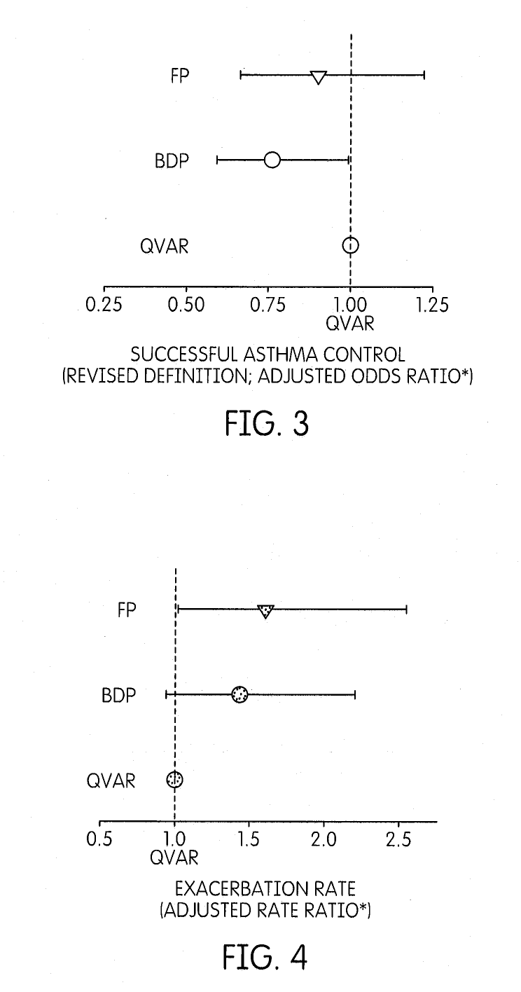

Predefined Proxy of Successful Asthma Control including SABA Use: In terms of revised definition of adjusted successful asthma control, Qvar® was significantly more effective than BDP (OR: 76; 95% CI: 0.59-0.99), and numerically more effective than FP (OR: 0.90; 95% CI: 0.66-1.22). See FIG. 3.

Exacerbation Rate: In terms of adjusted exacerbation rates, Qvar® was significantly more effective than FP (RR: 1.61; 95% CI: 1.02-2.55), and numerically more effective than BDP (RR: 1.44; 95% CI: 0.94-2.21). See FIG. 4.

What is claimed is:

1. An inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm, for maintaining lung function above 60% of baseline FEV₁, or reducing the occurrences of lung function falling below 60% of baseline FEV ₁.

2. An inhalable pharmaceutical solution aerosol as claimed in claim 1, comprising 0.02 to 0.6 percent by weight beclometasone dipropionate, 1 to 20 percent by weight ethanol and 80 to 99 percent by weight of propellant, wherein the percentages by weight are based on the total weight of the solution aerosol.

3. An inhalable pharmaceutical solution aerosol as claimed in claim 1, consisting essentially of beclometasone dipropionate, ethanol and a propellant selected from 1,1,1,2-tetrafluoroethane, 1,1,1,2,3,3,3-heptafluoropropane and a mixture thereof.

4. An inhalable pharmaceutical solution aerosol as claimed in claim 1, wherein the aerosol has a droplet size having a mass median aerodynamic diameter of 0.8-1.2 μm.

5. An inhalable pharmaceutical solution aerosol as claimed in claim 1, for maintaining lung function above 70% of baseline FEV₁, or reducing the occurrences of lung function falling below 70% of baseline FEV₁.

6. An inhalable pharmaceutical solution aerosol as claimed in claim 1, for a patient showing insufficient response to inhalable fluticasone propionate and/or an inhalable formulation comprising beclometasone dipropionate and a chlorofluorocarbon propellant.

7. A method for maintaining lung function above 60% of baseline FEV₁, or reducing the occurrences of lung function falling below 60% of baseline FEV₁ by administering to a patient in need thereof, an inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm.
